(19) United States
(12) Patent Application Publication
(10) Pub. No.: US 2012/0185053 A1
(43) Pub. Date: Jul. 19, 2012

(54) Spacer Apparatus and Method for Achieving Improved Fit and Balance in Knee Joints

(76) Inventor: Richard Berger, Chicago, IL (US)

(21) Appl. No.: 13/303,481

(22) Filed: Nov. 23, 2011

Related U.S. Application Data
(60) Provisional application No. 61/416,355, filed on Nov. 23, 2010.

(51) Int. Cl. A61F 2/38

(52) U.S. Cl. 623/20.16

(57) ABSTRACT

A spacer apparatus and method of using the same can be employed to achieve improved fit and balance in a knee joint in knee arthoplasty without requiring multiple cuts to the distal femur or proximal tibia. The spacer apparatus is composed of a biocompatible material in a lattice structure. The spacer apparatus can be pre-selected or pre-shaped, or can be selected or shaped at the time of use to have a thickness and shape appropriate to be used as a spacer to improve knee fit, or as a shim to improve knee balance. The spacer apparatus can be used in connection with a femoral implant, a tibial implant, or both. The spacer apparatus can be shaped to alter the orientation of a femoral or tibial implant in the varus/valgus orientation, the anterior/posterior orientation, or both. The spacer apparatus can be used singly or in combination with other spacer implants to improve fit and balance.

### CROSS-REFERENCES TO RELATED APPLICATIONS

This application claims priority to provisional application Ser. No. 61/416,355, filed on Nov. 23, 2010 by Dr. Richard Berger. This application is related to copending PCT application “Spacer Apparatus and Method For Achieving Improved Fit and Balance in Knee Joints filed on the same day as the present application by Dr. Richard Berger. All of these applications are incorporated herein by reference.

### Statement Regarding Federally Sponsored Research or Development: Not applicable Background

Knee replacement surgery, also known as knee arthoplasty, is an important course of treatment for a number of problems that can occur with respect to the knee joint. Knee arthoplasty can be used as a treatment modality for chronic knee pain and various knee dysfunctions, including arthritis. Knee arthoplasty can be necessitated by acute injuries, as well as chronic or degenerative conditions.

The knee joint is generally defined as the point of articulation of the femur with the tibia. The knee joint consists of bony structures, primarily including the distal femur, the proximal tibia, and the patella. The knee also contains soft tissue and ligaments within and surrounding these structures, the primary purpose of which is to provide stability of the joint and to provide a shock-absorbing cushion between the distal femur and the proximal tibia.

A number of conditions or injuries can cause deterioration or dysfunction resulting in direct contact between the distal femur and proximal tibia. Such direct contact results in significant pain and reduced function. One of the purposes of knee arthoplasty is to replace knee structures, particularly the distal end of the femur and/or the proximal end of the tibia, with prosthetic replacement structures, known as implants, to re-establish a stable, balanced joint capable of smooth, pain-free movement.

Knee arthoplasty often involves work on all three bony structures within the knee. One step, resurfacing of the patella, is relatively easy to accomplish and is often performed in a single step. A significant portion of the remainder of the knee arthoplasty procedure is preparation of the distal femur and proximal tibia to receive femoral and tibial implants, respectively. This preparation typically requires a number of precise cuts to the distal femur and proximal tibia.

One of the existing challenges of knee arthoplasty is fitting femoral and tibial implants to the femur and tibia, respectively, in such a manner that the post-arthoplasty knee joint is not too loose, and is balanced in both the varus/valgus and the anterior/posterior orientations. Appropriate fit and balance are important to the stability and range of motion of the replaced knee joint, and also play a significant role in the durability of the implants and the outcomes experienced by the patient, including range of motion and pain reduction.

One technique for attempting to achieve appropriate fit and balance is to position the tibial and femoral implants in an optimal orientation with respect to each other and to the distal femur and proximal tibia, such orientation being achieved by cutting the distal femur and proximal tibia at angles designed to produce appropriate fit and balance within the knee joint once the implants are placed in connection with the prepared bone surfaces. This technique has several disadvantages. It is often challenging to predict the correct angles and depths of cutting required prior to fitting the implants. Because the fit and balance of the implants relies on the angle and depth of cuts to the tibial and femoral bones, the surgeon is often required to make multiple cuts to these bones prior to finalizing the placement of the implants. Increased cutting results in greater trauma to the patient, longer recovery periods, and a reduced chance of an optimal outcome if subsequent arthoplasty is needed on the same knee joint. Moreover, errors in judgment or execution sometimes cannot be corrected after such cuts are made.

Several techniques have developed to attempt to mitigate these disadvantages. For example, the technique described in U.S. Pat. No. 5,733,292 involves the use of adjustable trial prosthesis components to help assess the accuracy and appropriateness of cuts prior to final fitting of the implants. Measuring devices, such as that described in U.S. Pat. 7,578,821, attempt to provide the surgeon with more detailed information pertinent to appropriate placement of the tibial and femoral implants, thus attempting to reduce the number of required cuts.

Although some of the techniques and devices described above have improved outcomes for knee arthoplasty, currently available devices and techniques still suffer a number of disadvantages. Currently available devices and techniques do not allow a surgeon performing knee arthoplasty to place tibial and femoral implants reliably so as to achieve proper fit and balance without making multiple trial-and-error cuts to the femur or tibia.

A need exists for new apparatuses and methods for reliably achieving fit and balance in knee joints undergoing knee arthoplasty without a need for multiple cuts to the femur or tibia. Ideally, such apparatuses and methods would permit a surgeon to make adjustments achieve proper fit and balance within the knee joint without requiring multiple cuts to the distal femur or proximal tibia. Such apparatuses and methods would, ideally, be useable in conjunction with a variety of currently-existing arthoplasty devices such as cutting guides, saw blades, and measurement systems. At least some of these objectives are met by the versions of the present invention.

### SUMMARY

The present invention is directed to an apparatus and method that satisfies the need for new apparatuses and methods for reliably achieving improved fit and balance in knee joints undergoing knee arthoplasty without a need for multiple cuts to the femur or tibia. In one version, the present invention is directed to a spacer apparatus used to alter the spacing and angle of orientation of femoral or tibial implants connected to the distal femur or proximal tibia, respectively, for the purpose of achieving better fit and balance in the knee joint. The spacer apparatus is a lattice made of a biocompatible material sufficiently rigid to hold the implant in the desired position and orientation. Preferably, this material is hardened PMMA. In another version, the present invention is further directed to a method of using a spacer to achieve improved fit and balance in a knee joint undergoing knee arthoplasty by altering the spacing and angle of orientation of femoral or tibial implants connected to the distal femur or proximal tibia, respectively.

A spacer apparatus can be shaped in varying thicknesses and shapes to permit alteration of the fit or balance of tibial or femoral implants. The thickness and shape of a spacer apparatus can be pre-shaped prior to a knee revision procedure, or, alternatively, a spacer apparatus of desired thickness and shape can be shaped at the time of use by, for example, cutting a spacer apparatus of desired thickness and shape from a sheet of spacer material.

In one version of the invention, a user selects and places a spacer of desired thickness and shape in connection with a femoral implant and distal femur so as to achieve desired fit and balance in the knee joint. In another version of the invention, a user selects and places a spacer of desired thickness and shape in connection with a tibial implant and proximal tibia so as to achieve the desired fit and balance of the knee joint. In another version of the invention, a user selects and places multiple spacers of desired thicknesses and shapes in connection with both a femoral implant and distal femur and a tibial implant and proximal tibia so as to achieve the desired fit and balance of the knee joint.

A spacer apparatus can be used to achieve the desired fit of a knee joint by acting as a spacer that increases the distance between an implant and a bone surface to which the implant is connected. In one version of the invention, a spacer apparatus acts as a spacer between a femoral implant and distal femur. In another version of the invention, a spacer apparatus acts as a spacer between a tibial implant and proximal tibia. In another version of the invention, spacer apparatuses act as spacers between both the femoral implant and distal femur and, separately, the tibial implant and proximal tibia. The magnitude of the spacing function of a spacer apparatus of versions of the present invention can be determined either by selecting a spacer of desired thickness or by stacking multiple spacers to achieve a desired thickness.

A spacer apparatus can also be used to achieve improved balance in a knee joint by acting as a shim that changes the angle of interface between an implant and the bone surface with which it is connected. In one version of the invention, a spacer apparatus is used to alter the angle of the interface between a femoral implant and the distal femur in the varus/valgus orientation, the anterior/posterior orientation, or both. In another version of the invention, a spacer apparatus is used to alter the angle of the interface between a tibial implant and the proximal tibia in the varus/valgus orientation, the anterior/posterior orientation, or both. In another version of the invention, multiple spacer apparatuses are used to alter the angle of the interface between the femoral implant and the distal femur in the varus/valgus orientation, the anterior/posterior orientation, or both, and independently, to alter the angle of the interface between a tibial implant and the proximal tibia in the varus/valgus orientation, the anterior/posterior orientation, or both. In another version of the invention, a spacer apparatus is used as a shim to alter the angle of interface of the femoral implant and/or tibial implant while simultaneously acting as a spacer to increase the distance between the femoral implant and distal femur and/or tibial implant and proximal tibia.

Another version of the invention is directed to a method for using a spacer apparatus to produce desired fit and balance in a knee joint in knee arthoplasty. As related to primary knee surgery, in one version of the invention, a surgeon prepares the knee to receive both a femoral and a tibial implant. The surgeon then evaluates whether the distance or the angle of interface for either or both of the implants is desired to be altered to achieve better fit or balance of the knee joint. The surgeon then uses one or more spacer apparatuses to alter the spacing or angle of interface of either or both of the femoral or tibial implants.

The method of the versions of this invention may also be used in connection with revision surgery. As related to revision surgery, in one version of the invention, a surgeon prepares the knee to receive a femoral implant. The surgeon then evaluates whether the distance between the femoral implant and distal femur or the angle of interface between the femoral implant and distal femur are desired to be altered to achieve better fit or balance of the knee joint. The surgeon uses one or more spacer apparatuses to alter the spacing or angle of interface of the femoral implant as desired. In another version of the invention, a surgeon prepares the knee to receive a tibial implant. The surgeon then evaluates whether the distance between the tibial implant and proximal tibia or the angle of interface between the tibial implant and proximal tibia are desired to be altered to achieve better fit or balance of the knee joint. The surgeon uses one or more spacer apparatuses to alter the spacing or angle of interface of the tibial implant as desired.

Multiple versions of the invention can be used in combination or concert with each other, and with other known techniques, to achieve desired fit and balance in the knee joint. Specifically, multiple versions of the invention can be used simultaneously in femoral and tibial implants in the same knee joint. Multiple versions of this invention can also be used simultaneously in which a spacer apparatus acts as a spacer and in which the same or a different spacer apparatus acts as a shim. Multiple versions of this invention can also be used simultaneously in which a spacer apparatus alters the angle of interface between an implant and the bone surface in the varus/valgus orientation and in which the same or a different spacer apparatus alters the angle of interface between an implant and the bone surface in the anterior/posterior orientation.

The versions of the present invention, as described above, solve problems currently known to the art by providing the ability to alter the fit and balance of a knee joint undergoing knee arthoplasty without the need for multiple cuts to the distal femur or proximal tibia. The versions of the present invention further enable an efficient means for fitting implants without expensive measurement or fitting equipment or use of multiple trial implants.

In the Summary above and in the Description, and the claims below, and in the accompanying drawings, reference is made to particular features of the invention. It is to be understood that the disclosure of the invention in this specification includes all possible combinations of such particular features.

### BRIEF DESCRIPTION OF THE DRAWINGS

These and other features, aspects, and advantages of the present invention will become better understood with regard to the following description and accompanying drawings, where:

FIG. 1 shows a lattice structure for various versions of the invention, which such lattice can be of varying thickness and shaped to a desired shape prior to or during knee arthoplasty.

FIG. 2 shows a top view of a lattice structure for various versions of the invention;

FIG. 3 shows a side view of spacer apparatuses of varying thicknesses according to versions of the invention;

FIG. 4 shows a perspective view of a spacer apparatus in one shape appropriate for use in connection with a tibial implant according to a version of the invention;

FIG. 5 shows a perspective view of a spacer apparatus of multiple spacers in shapes appropriate for use in connection with a femoral implant according to a version of the invention;

FIG. 6 shows a side view of a spacer apparatus shaped to alter the angle of interface of a tibial implant in the varus/valgus orientation according to a version of the invention;

FIG. 7 shows a side view of a spacer apparatus shaped to alter the angle of interface of a tibial implant in the anterior/posterior orientation according to a version of the invention;

FIG. 8 shows an exploded view of a spacer apparatus placed in connection with a tibial implant and a tibia prepared to receive a tibial implant according to a version of the invention;

FIG. 9 shows an exploded view of a spacer apparatus shaped to alter the angle of an implant in the varus/valgus orientation in connection with a tibial implant and a tibia prepared to receive a tibial implant according to a version of the invention;

FIG. 10 shows an exploded view of a spacer apparatus shaped to alter the angle of an implant in the posterior/anterior orientation in connection with a tibial implant and a tibia prepared to receive a tibial implant according to a version of the invention;

FIG. 11 shows an exploded view of a spacer apparatus placed in connection with a femoral implant and a femur prepared to receive a femoral implant according to a version of the invention;

FIG. 12 shows an exploded view of a spacer apparatus shaped to alter the angle of an implant in the varus/valgus orientation in connection with a femoral implant and a femur prepared to receive a femoral implant according to a version of the invention;

FIG. 13 shows an exploded view of a spacer apparatus shaped to alter the angle of an implant in the anterior/posterior orientation in connection with a femoral implant and a femur prepared to receive a femoral implant according to a version of the invention;

FIG. 14 shows an exploded view of a version of a femur prepared to receive a femoral implant;

FIG. 15 shows an exploded view of a version of a tibia prepared to receive a tibial implant;

FIG. 16 shows a perspective view of a spacer apparatus in connection with a femur and femoral implant according to a version of the invention;

FIG. 17 shows a perspective view of a spacer apparatus in connection with a tibia and tibial implant according to a version of the invention;

FIG. 18 shows an exploded view of a spacer apparatus affixed to a tibia by fasteners according to a version of the invention.

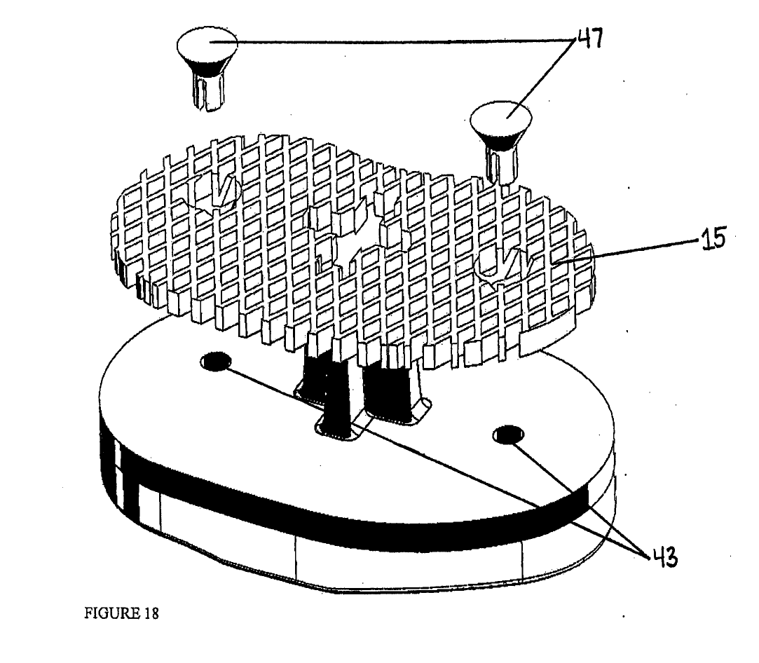

### DESCRIPTION

The versions of the present invention are directed towards a spacer apparatus for use in achieving improved fit and balance in a knee joint in knee arthoplasty, and methods for using a spacer apparatus to achieve improved fit and balance in a knee joint in knee arthoplasty.

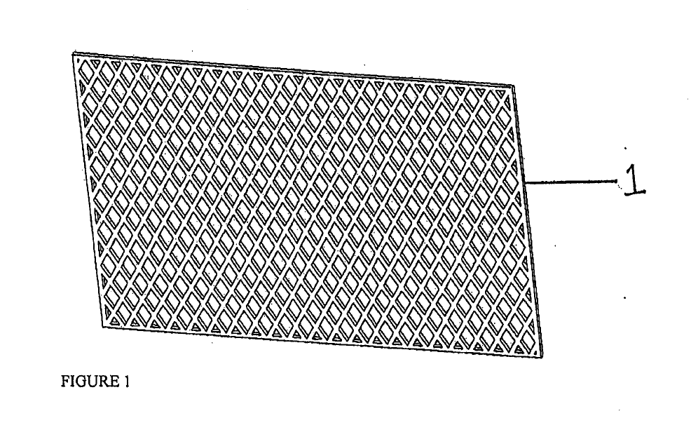

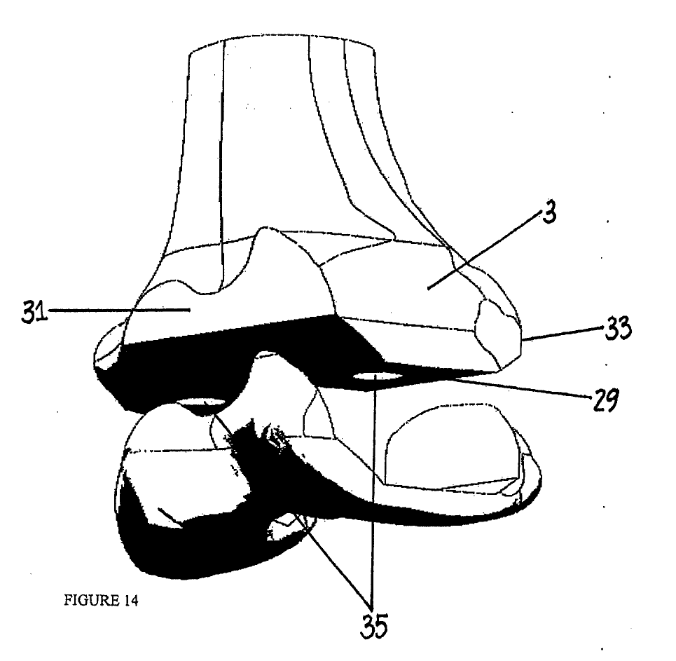

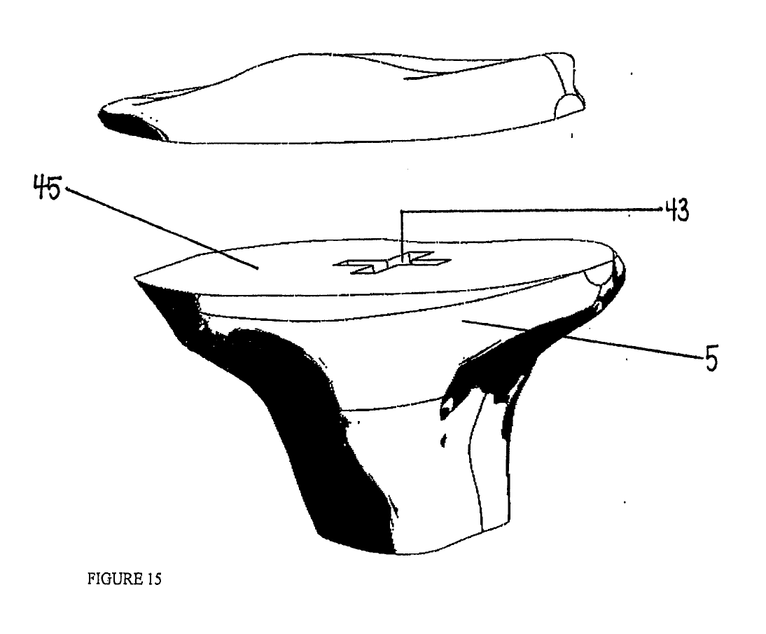

A “spacer” according to the versions of the present invention is an apparatus shaped from a lattice structure [1] as shown in FIG. 1, for use in knee arthoplasty for altering: (1) the distance between a femoral [11] or tibial implant [13] and the prepared bone surfaces of the distal femur [3], as shown in FIG. 14, or proximal tibia [5], as shown in FIG. 15; (2) the tightness or looseness of the knee joint after a distal femur [3] or proximal tibia [5] is prepared to receive a femoral [11] or tibial implant [13]; or (3) the angle of interface between a femoral [11] or tibial implant [13] and the surface of the distal femur [3] or proximal tibia [5].

A spacer can be composed of any biocompatible material that is sufficiently rigid to prevent excessive movement of the tibial or femoral implants after the spacer is placed in connection therewith. Optionally, a biocompatible material can be selected that provides a desired degree of rigidity to hold the knee implants in the correct position while bone cement or other connective substance sets, hardens, or is placed. Suitable biocompatible materials include, but are not limited to PTFE, ePTFE, other fluropolymers, polyolefin rubber, PET, EVA, or polypropylene, and, preferably, hardened PMMA. While the versions of the spacer apparatus depicted and described in specific examples herein are composed of hardened PMMA, other suitable biocompatible materials, including those listed above, are within the scope of the versions of this invention.

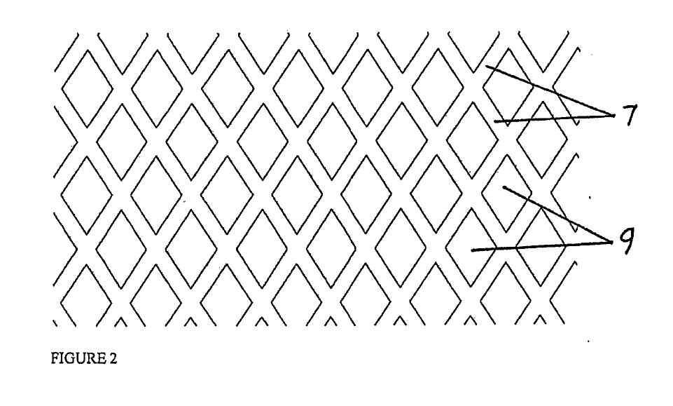

A spacer apparatus has a lattice structure comprised of arms [7] and pores [9], a version of which is shown in FIG. 2, Pores [9] can be any shape or size, provided: (a) that the spacer apparatus has sufficient rigidity to prevent excessive movement of the tibial or femoral implants; and (b) that the pores can be infiltrated by bone cement or other connective substances commonly used to affix bone implants to bone. In one version of the invention, pores [9] are diamond-shaped. In another version of the invention, pores [9] have a size of approximately 6 millimeters by approximately 3 millimeters, the measurements taken across the long and short axes of a single pore [9]. Other pore [9] shapes and sizes may be used within the scope of the invention.

Arms [7] of the spacer apparatus lattice can be any shape or size that provides sufficient rigidity to prevent excessive movement of the tibial or femoral implants. In one version of the invention, arms [7] are each approximately 1 millimeter in width, as shown in FIG. 1. Other lattice arm [7] sizes may be used within the scope of the invention.

A spacer according to the versions of this invention can be used to achieve desired fit or balance of the knee joint. “Fit” of the knee joint according to the versions of this invention is the suitability of the interface between a femoral implant [11] and tibial implant [13] determined by the overall distance between the distal end of the femoral implant [11] and the distal femur [3] and/or the overall distance between the proximal end of the tibial implant [13] and the proximal tibia [5] in light of the tension provided by the connective tissue and ligaments of the knee. “Looseness” occurs when the fit of the knee joint is not suitable for desired function of the knee joint, and particularly when the femoral implant [11] and tibial implant [13] are not sufficiently sized and/or located/oriented relative to the overall anatomy to create desired tension in the connective tissue and ligaments of the knee.

“Balance” of the knee joint is the suitability of the interface between a femoral implant [11] and tibial implant [13] to allow desired function of the knee joint, determined by the angle of interface between a femoral implant [11] and distal femur [3] and the angle of interface between the tibial implant [13] and proximal tibia [5] in light of the tension provided by the connective tissue and ligaments of the knee. “Imbalance” occurs when the angle of interface between the faces of the femoral and tibial implants is not suitable for desired function of the knee joint. Imbalance can occur in the varus/valgus orientation, the anterior/posterior orientation, or both. Fit and balance are “improved” when the use of one or more spacer apparatuses, or methods employing the same, reduces or eliminate looseness, imbalance, or both.

A “bone surface” is the surface of a knee joint bone that that has been prepared to receive an implant in a knee arthoplasty procedure. Bone surfaces preferably include prepared surfaces of the distal femur, the proximal tibia, or both.

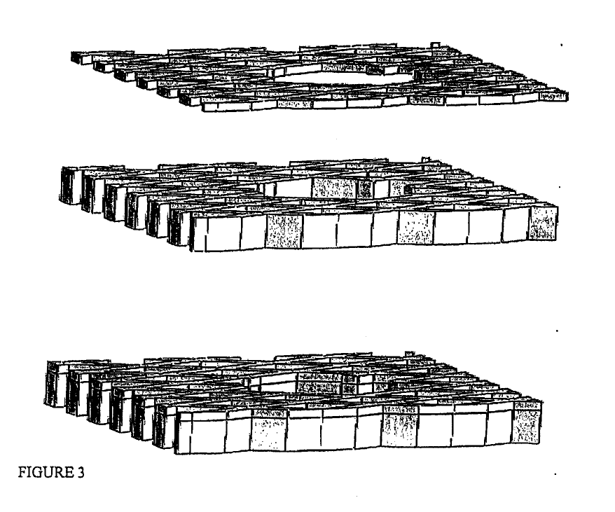

“Thickness” according to the versions of the present invention is the dimension of a spacer apparatus measured from one face of the lattice to the other face, as shown in FIG. 3. A spacer can be of varying thicknesses in the range of approximately 1 millimeter to 30 millimeters, and preferably of 1 millimeter to 10 millimeters, as desired, to alter the distance or angle of interface of an implant with the distal femur or proximal tibia, or to alter the fit between an implant and the distal femur or proximal tibia. Thicknesses below this range may lack sufficient rigidity to prevent excessive movement of the implants, while thicknesses above this range are not typically required for knee arthroplasty or may result in insufficient strength of connective substances such as bone cement. Desired thickness can be achieved by selecting or shaping a single spacer to desired thickness, or by stacking multiple spacers.

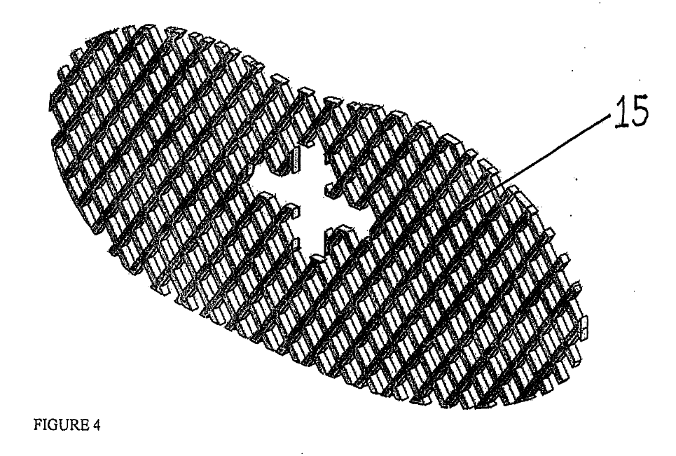

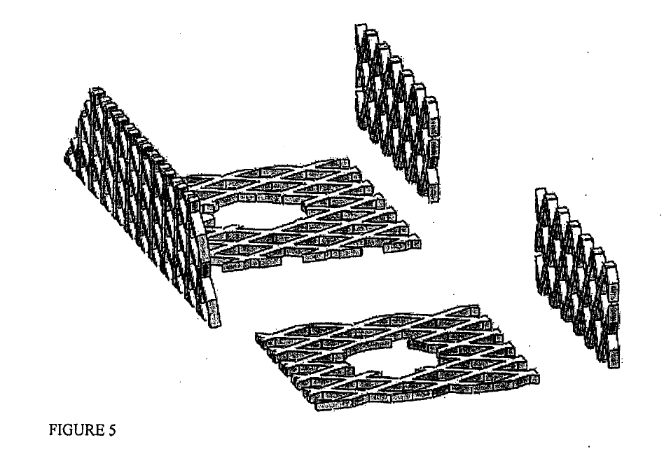

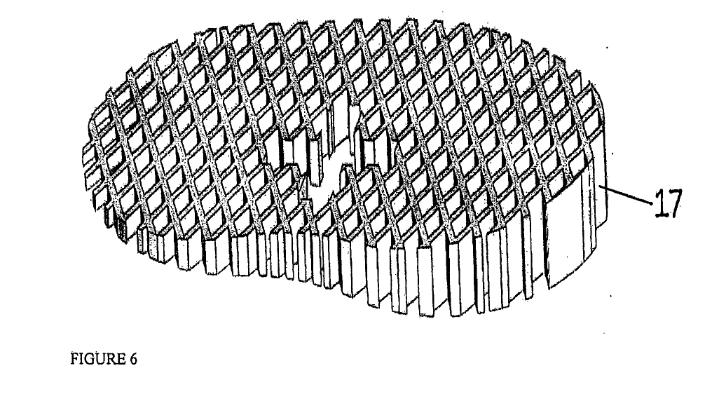

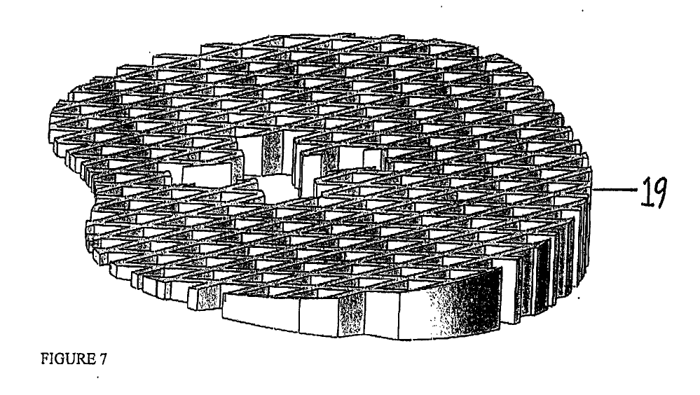

The thickness of a spacer can be uniform, as shown in FIGS. 4 and 5. Alternatively, the thickness of a spacer can be variable to enable a spacer to act simultaneously as a shim to achieve improved fit by changing the angle of interface between an implant and a bone surface, versions of which are shown in FIGS. 6 and 7.

The thickness of a spacer may optionally vary across a single spacer to change the angle of interface in the varus/valgus orientation, a version of which is shown in FIG. 6. The thickness of a spacer may optionally vary across a single spacer to change the angle of interface in the anterior/posterior orientation, as shown in FIG. 7. Optionally, thickness may vary across a single spacer to change the angle of interface in the varus/valgus and anterior/posterior orientations simultaneously. Optionally, multiple spacers are used simultaneously to achieve greater thickness or to achieve variable thickness in one or more orientations, as desired.

A spacer apparatus according to the versions of this invention may have a desired shape. “Shape” according to the versions of this invention means the three-dimensional surface contour of a spacer apparatus. The shape of a spacer can be shaped prior to a knee arthoplasty procedure or, alternatively can be shaped by a surgeon or other end user during a knee arthoplasty procedure. In one version of the invention, a spacer is shaped by the user during a knee arthoplasty procedure by cutting the spacer to a desired shape from a lattice structure [1] of spacer material of desired thickness. Optionally, multiple spacers may be shaped by the user during a knee arthoplasty procedure by cutting the spacers to a desired shape from a lattice structure [1] of spacer material and stacking said spacers to achieve the desired thickness, angle or orientation, or both.

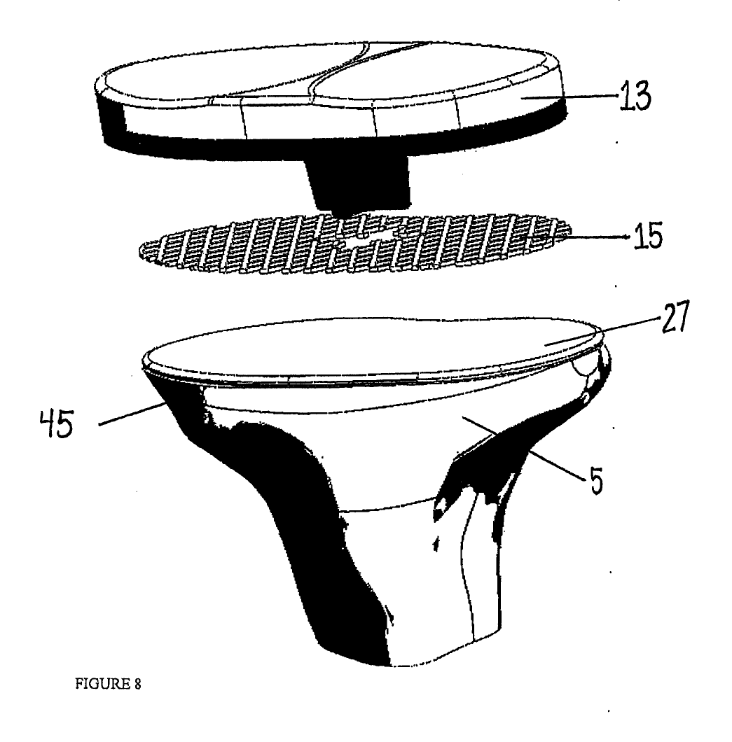

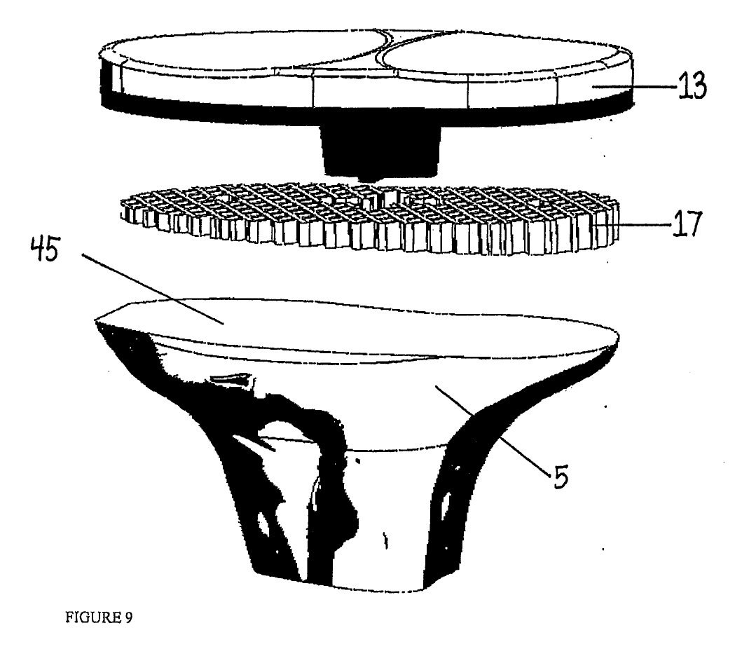

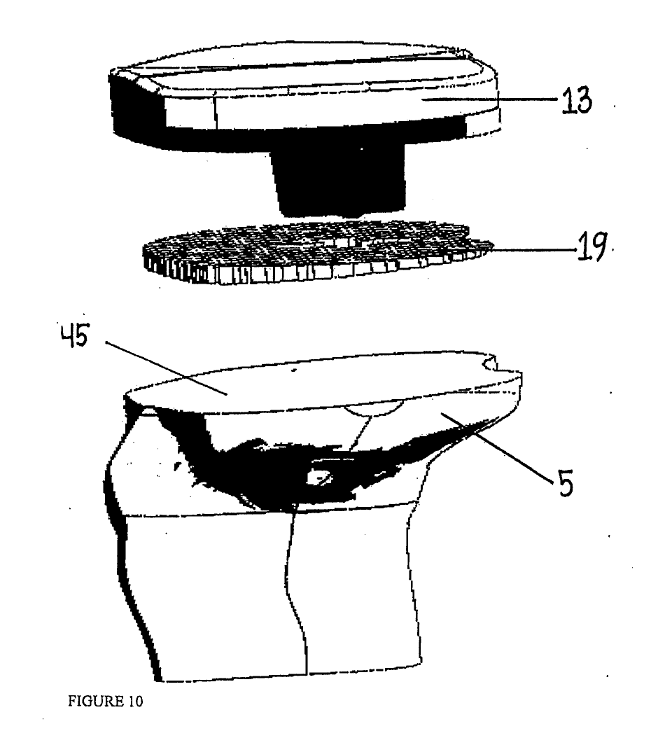

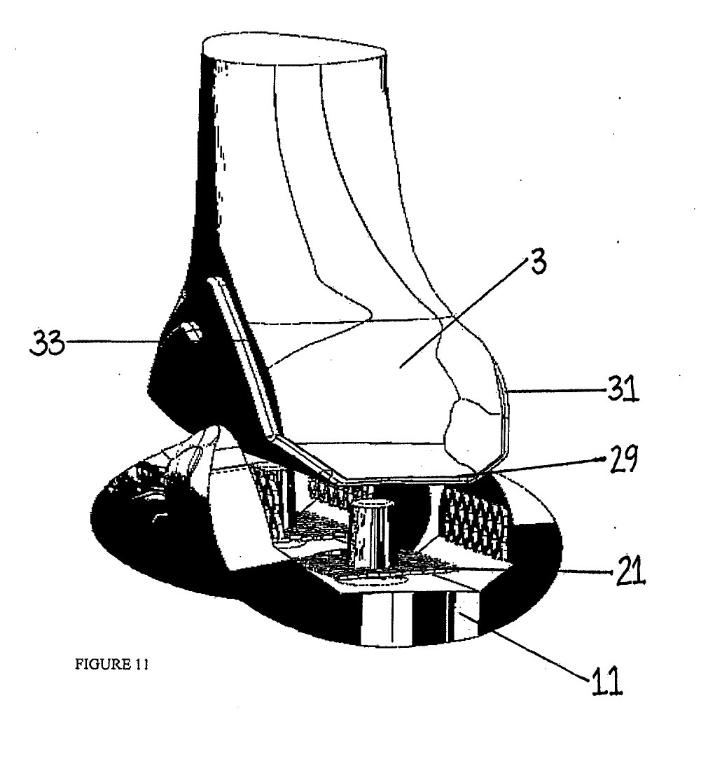

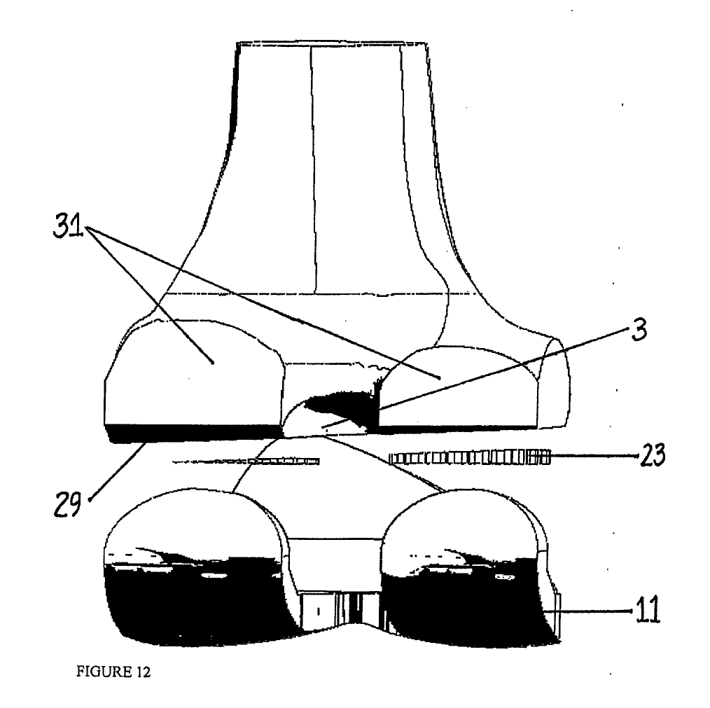

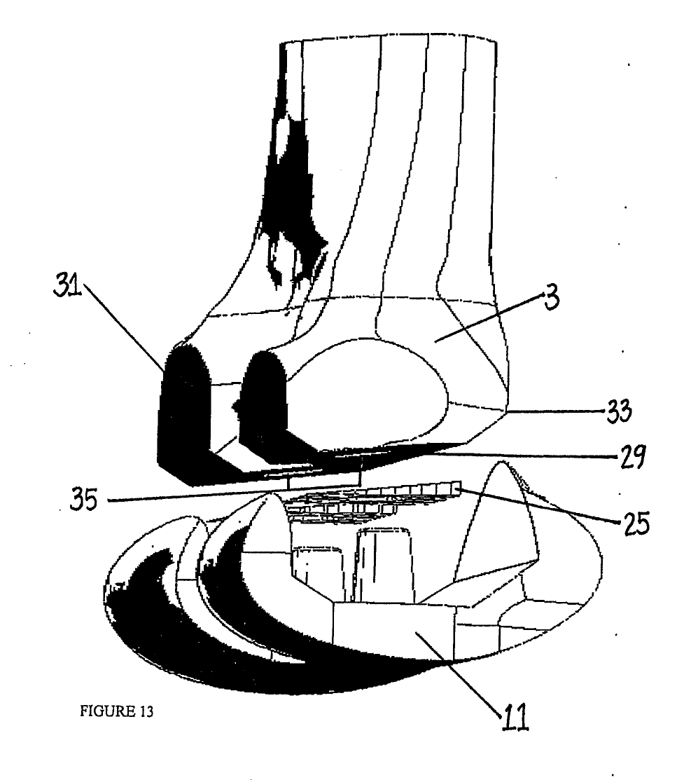

In another version of this invention, a spacer is of desired shape is selected prior to a knee arthoplasty procedure. Suitable shapes are those appropriate to achieve the desired fit or balance of the knee joint when used in connection with a femoral implant [11], a tibial implant [13], or both. Several shapes appropriate for versions of the invention are a spacer apparatus of uniform thickness for a tibial implant [15], as shown in FIG. 8, a spacer apparatus shaped to alter varus/valgus orientation in a tibial implant [17], as shown in FIG. 9, a spacer apparatus shaped to alter anterior/posterior orientation in a tibial implant [19], as shown in FIG. 10, a spacer apparatus of uniform thickness for a femoral implant [21], as shown in FIG. 11, a spacer apparatus shaped to alter varus/valgus orientation in a femoral implant [23], as shown in FIG. 12, a spacer apparatus shaped to alter anterior/posterior orientation in a femoral implant [25], as shown in FIG. 13.

A spacer apparatus of desired shape can be used to achieve desired balance of the knee joint by acting as a “shim” to change the angle of interface between the femoral implant [11] and distal femur [3] or to change the angle of interface between the tibial implant [13] and proximal tibia [5]. According to the versions of this invention, the angle of interface of either or both of a femoral [11] or tibial implant [13] can be changed in the varus/valgus orientation, as shown in FIGS. 9 and 12, or in the anterior/posterior orientation, as shown in FIGS. 10 and 13, or both orientations simultaneously.

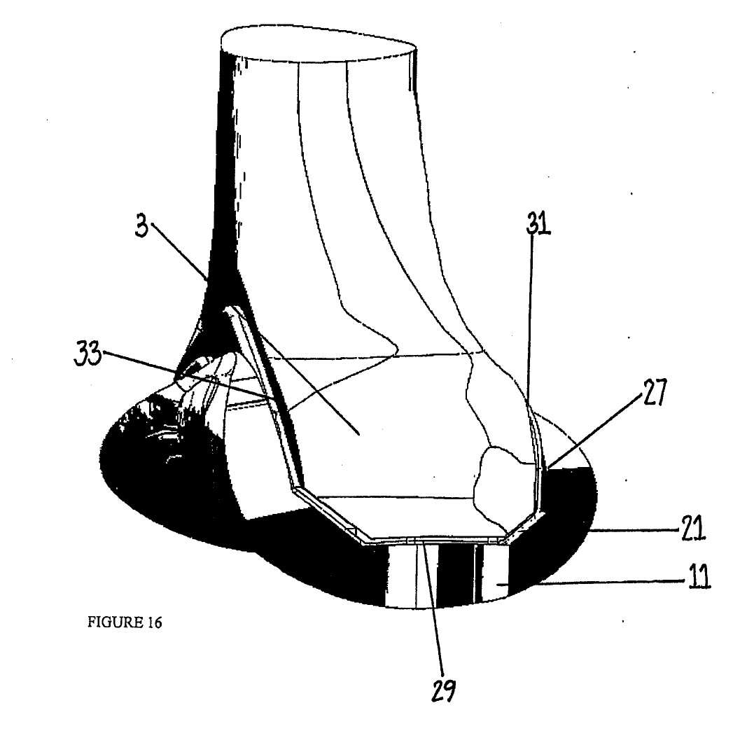

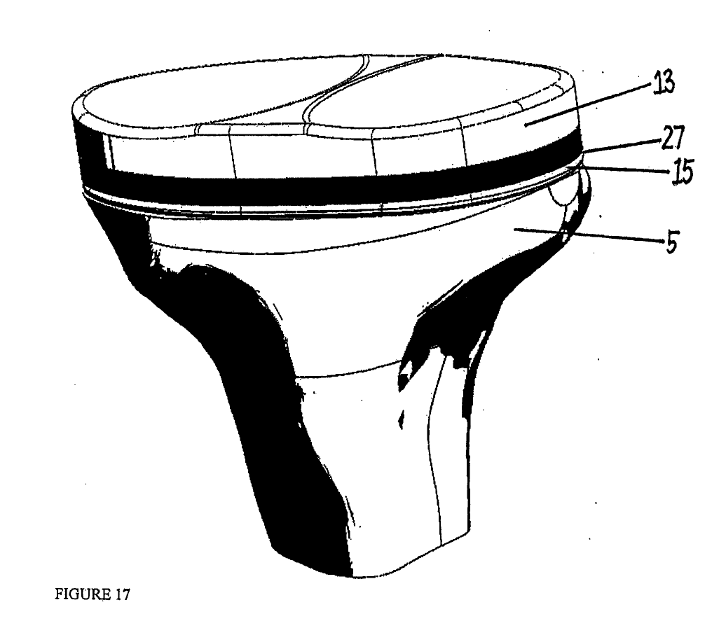

A spacer apparatus of the versions of the present invention can be placed into contact or connection with both an implant and a bone surface prepared to receive an implant, versions of which are shown in FIGS. 16 and 17. “Contact” or “connection” according to the present invention is either direct physical interface with an implant and/or a bone surface, or, optionally, indirect interface with an implant and/or bone surface, in which an intermediary substance [27] is interposed between the spacer and the implant and/or bone surface. The intermediary substance [27] can be any substance appropriate for use in knee arthoplasty, such as cancellous bone paste, or, preferably, bone cement.

Use of the versions of this invention in connection with a femoral implant involves resection of the distal femur in a manner appropriate for knee arthoplasty. Such resection typically involves cutting or refacing the distal [29], posterior [31], and anterior [33] faces of the distal femur [3], as shown in FIG. 14. Resection can also include drilling holes [35] or creating a cavity of a shape appropriate to receive a femoral implant [11], as shown in FIG. 14. After resection, a femoral implant [11] can be fitted to a distal femur [3], versions of which are shown in FIGS. 12, 13, and 16. In the versions of this invention, one or more spacers can be placed between the distal face [29] of the femur [3] and the femoral implant [11], between the posterior face [31] of the distal femur [3] and the femoral implant [11], or between the anterior face [33] of the distal femur [3] and the femoral implant [11], FIG. 11 demonstrates a version of placement of spacer apparatuses at the distal [29], posterior [31], and anterior [33] faces of the distal femur [3]. Placement of one or more spacer apparatus at any one or any combination of these positions, or other positions, is encompassed by the versions of this invention. The use of multiple spacer apparatuses at any one or any combination of these positions, or other positions, is also encompassed by the versions of this invention.

Use of the versions of this invention in connection with a tibial implant [13] involves resection of the proximal tibia [5] in a manner appropriate for knee arthoplasty. Such resection typically involves cutting or resecting the proximal tibia [5], as shown in FIG. 15. Resection can also include drilling holes [43] or creating a cavity of a shape appropriate to receive a tibial implant, as shown in FIG. 15. After resection, a tibial implant [13] can be fitted to the proximal tibia [5], versions of which are shown in FIGS. 8, 9, and 10. In the versions of this invention, one or more spacers can be placed between the proximal face [45] of the proximal tibia [5] and the tibial implant [13]. Placement of one or more spacer apparatus at other positions is encompassed by the versions of this invention. The use of multiple spacer apparatuses at this or other positions is also encompassed by the versions of this invention.

A spacer apparatus may optionally be connected in unaffixed contact with the femoral [11] or tibial [13] implants. Optionally, a spacer apparatus may be in unaffixed contact with the prepared bone surface of the femur [3] or tibia [5]. A spacer apparatus may optionally be affixed to one or more of the femoral [11] or tibial [13] implants by one or more fasteners [47]. A spacer apparatus may optionally be affixed to the prepared bone surface of one or more of the femur [3] or tibia [5] by one or more fasteners [47]. Such fasteners [47] can be of any type appropriate for use in knee arthoplasty. Examples of acceptable fasteners include pins, posts, keel-type connections, staples, glues, buttons, friction fasteners, sutures, hooks, and, preferably, cements.

The versions of this invention encompass methods of using one or more spacer apparatus as described herein to improve one or more of fit or balance in a knee joint undergoing knee arthoplasty. A distal femur [3] or proximal tibia [5] are resected in a manner appropriate for knee arthoplasty, versions of which are shown in FIGS. 14 and 15. One or more spacers are selected or shaped for thickness and shape appropriate to improve any looseness or imbalance in the knee joint. These spacers are placed in connection with one or more of: (1) femoral implant [11] and distal femur [3]; (2) the tibial implant [13] and proximal tibia [5]; or (3) both. Optionally, the user can select or shape the spacer to the desired shape during the knee arthoplasty procedure, as described herein. Optionally, multiple spacers can be used to create a desired shape or thickness, as described herein.

The versions of this invention encompass methods for improving the fit of the knee joint in knee arthoplasty as well as methods for improving the balance of the knee joint in knee arthoplasty. The versions of this invention further encompass methods for both primary knee procedures and revision procedures.

It should be noted that the phrase “step of” as implemented in the claims below is distinct from, and not intended to mean, “step for” as that phrase is used in 35 U.S.C. §112 ¶6.

Although the present invention has been described in considerable detail with reference to certain preferred versions thereof, other versions are possible. For example, other materials, pore sizes, lattice arm sizes, thicknesses, or shapes may be used for a spacer apparatus other than those described in detail. Similarly, other placements of a spacer apparatus may be employed than those shown in detail. Similarly, other steps may be included, or omitted from, the methods of the versions of this invention. For example, trial implants need not be used before assessing the fit and balance of a knee joint. Therefore, the spirit and scope of the claims should not be limited to the description of the preferred versions described herein.

What is claimed is:

1: A spacer apparatus comprising a lattice composed of a rigid biocompatible material, said spacer apparatus selected or shaped to have a shape and thickness whereby at least one of:
(a) looseness;
(b) varus/valgus imbalance; or
(c) anterior/posterior imbalance
will be improved when said spacer apparatus is placed in connection with a knee implant and a bone surface prepared to receive said implant in knee arthoplasty.

2: The spacer apparatus of claim 1, in which said spacer is shaped for connection with at least one of:
(a) a tibial implant and a proximal tibia bone surface prepared to receive said tibial implant; or
(b) a femoral implant and distal femur bone surface prepared to receive said femoral implant.

3: The spacer apparatus of claim 2, in which said rigid biocompatible material is hardened PMMA.

4: The spacer apparatus of claim 2, in which at least one spacer is connected to the distal face of the distal femur bone surface and a femoral implant.

5: The spacer apparatus of claim 2, in which at least one spacer is connected to the anterior face of the distal femur bone surface and a femoral implant.

6: The spacer apparatus of claim 2, in which at least one spacer is connected to the posterior face of the distal femur bone surface and a femoral implant.

7: The spacer apparatus of claim 2, in which at least one spacer is connected to at least one of
(a) a bone surface prepared to receive an implant; and
(b) an implant by a means for fastening.

8: The spacer apparatus of claim 7, in which said means for fastening is bone cement.

9: The spacer apparatus of claim 1, in which said spacer has a thickness at least one of the following ranges:
(a) approximately one to approximately thirty millimeters; or
(b) approximately one to approximately ten millimeters.

10: The spacer apparatus of claim 1, in which said lattice contains pores, and said pores have an opening shaped substantially as a diamond.

11: The spacer apparatus of claim 9, in which said pores have an opening area of approximately 18 square millimeters.

12: The spacer apparatus of claim 1, in which said lattice contains arms, and said arms have a width of approximately one millimeter.

13: A method for improving fit and balance in a knee joint undergoing knee arthoplasty, said method comprising the steps of:
(a) resecting at least one bone surface within a knee in preparation for implantation of at least one implant;
(b) assessing said knee joint for fit and balance;
(c) selecting one or more spacer apparatuses; and
(d) placing said spacers in connection with at least one:
(i) implant; and
(ii) bone surface so as to improve fit and balance in said knee joint.

14: The method of claim 13, in which said rigid biocompatible material is hardened PMMA.

15: The method of claim 13, in which said spacer is selected or shaped to have a shape and thickness whereby at least one of:
(a) looseness;
(b) varus/valgus imbalance; or
(c) anterior/posterior imbalance will be improved when said spacer apparatus is placed in connection with a knee implant and a bone surface prepared to receive said implant.

16: The method of claim 15, in which multiple spacers are selected or shaped to have a collective shape and thickness whereby at least one of:
(a) looseness;
(b) varus/valgus imbalance; or
(c) anterior/posterior imbalance will be improved when said spacer apparatuses are placed in connection with one or more knee implants and one or more bone surfaces prepared to receive said implants.

17. The method of claim 13, in which the resected bone surface is one or more of:
(a) a proximal tibia; and
(b) a distal femur;
and in which said implant is one or more of:
(a) a tibial implant; and
(b) a femoral implant.

18: The method of claim 17, in which said spacer has a thickness at least one of the following ranges:
(a) approximately one to approximately thirty millimeters; or
(b) approximately one to approximately ten millimeters.

19. The method of claim 17, in which said lattice contains pores, and said pores have an opening shaped substantially as a diamond,

20. The method of claim 17, in which said pores have an opening area of approximately 18 square millimeters.

21. The method of claim 17, in which said lattice contains arms, and said arms have a width of approximately one millimeter.
